(19) United States
(12) Patent Application Publication
(10) Pub. No.: US 2013/0225964 A1
(43) Pub. Date: Aug. 29, 2013

(54) ELECTRODE FOR INTRAOPERATIVE NERVE STIMULATION

(71) Applicant: Andreas Langer, Waldkirch (DE)

(72) Inventor: Andreas Langer, Waldkirch (DE)

(73) Assignee: DR. LANGER MEDICAL GMBH, Waldkirch (DE)

(21) Appl. No.: 13/883,990

(22) PCT Filed: Nov. 7, 2011
(86) PCT No.: PCT/IB2011/054960

§ 371 Date: May 7, 2013

(30) Foreign Application Priority Data
Nov. 11, 2010 (DE) 10 2010 060 497.6

(51) Int. Cl. A61B 5/00

(52) U.S. Cl. CPC A61B 5/4848
USPC 600/377

(57) ABSTRACT

An electrode for intraoperative nerve stimulation of the vagus nerve, which electrode is suitable for continuous neuromonitoring during an operation and has an electrode holder with a strip-shaped design and made of an elastic material, and at least one electrically conductive contact surface, the latter being connectable via a signal-transmission medium to a signal transmitter and/or signal receiver and touching the vagus nerve on part of its circumference during the application. The electrode includes a first portion with a plastically deformable material such that, during the application, the electrode holder can be formed onto a vessel wall of a first blood vessel extending parallel to the vagus nerve and encompasses this blood vessel in order to affix the electrode in a hook-shaped fashion and by a second portion which, with an opposing curvature to that of the first portion can be placed around the vagus nerve in a loop-like fashion.

The invention relates to an electrode for intraoperative stimulation of the vagus nerve, having an electrode holder with a strip-shaped design and made of an elastic material, and at least one electrically conductive contact surface, the latter being connectable via a signal-transmission medium to a signal transmitter and/or signal receiver and touching the vagus nerve on part of its circumference during the application.

### BACKGROUND OF THE INVENTION

In order to be able to monitor during the operation whether, possibly, nerve structures situated in the operation region are being injured, intraoperative neuromonitoring has already been carried out during operations for a number of decades. The neuromonitoring technique is used in many types of surgical interventions, for example in neurosurgery, ear, nose and throat surgery, vessel surgery or thyroid surgery. Here, the nerve to be monitored is stimulated electrically and an action potential is formed, which can then, for example, be recorded in the form of electromyography signals on the effector organ of the relevant nerve, for example a muscle, via electrodes and analyzed. The recorded signal allows conclusions to be drawn about the functional state of the nerve.

Intraoperative neuromonitoring is of particular importance in, inter alia, thyroid surgery, where the recurrent laryngeal nerve can be injured, leading to voice disorders, loss of voice and/or respiratory or swallowing discomfort in the patient. In the case of an injury on both sides of the laryngeal nerve branching off the vagus nerve, there is even mortal danger to the patient during goiter operations.

As a result of the use of functional monitoring of nerves during the operation, it was also possible to reduce significantly the most common and most dangerous complication during thyroid surgery, namely the injury of the recurrent laryngeal nerve referred to as a recurrent paresis. However, as far as can be seen, use has so far practically only been made of intermittent methods, in which functional monitoring of the nerve can only be carried out before and after a dangerous action of the operator, by virtue of the nerve being stimulated by means of a manually guided electrode prior to and after an incision or the like. Here, the stimulation is carried out by means of stimulation probes, the structural design of which varies according to the manufacturer. By way of example, use is made of monopolar probes with spherical tips or bipolar embodiments with concentric pole tips or fork tips, or else use is also made of hook probes with optionally straight or bayonet-shaped shafts. Naturally, all these stimulation probes can only stimulate the nerve if they are manually placed onto the nerve to be stimulated by the operator.

Even if the intermittent neuromonitoring assists the operator in identifying an endangered nerve and in monitoring the nerve function during both thyroid surgery and other surgical interventions such as in neurosurgery, for example, this intermittent method does not provide secure protection to the endangered nerves because the function is only ever monitored prior to and after the per se risky surgical action. Thus, the intermittent monitoring does not prevent the surgeon from injuring a nerve or even severing the latter. Thus, a continuous monitoring method is sought after, which can be considered to be a type of early warning system for nerve damages. In this context, the term “continuously” should be understood to mean a stimulation of the relevant nerve taking place automatically, i.e. without manual action of the operator, in a non-stop fashion or at short time intervals and the recording of the response signal at the effector muscle.

On the other hand, so-called permanent electrodes, which are provided to remain indefinitely in the human body, are not suitable for intraoperative stimulation either since it is comparatively difficult to apply these and, in particular, these can only be removed by complicated interventions.

The document DE 10 2008 048 788 A1, by the applicant itself, discloses an electrode for intraoperative nerve stimulation, which is suitable for continuous neuromonitoring during an operation, in particular for neuromonitoring of the recurrent laryngeal nerve during thyroid operations. The electrode is characterized by an electrode body, which has an approximately T- or anchor-shaped design in cross-section and has an electrode shaft and at least one holding bracket which protrudes on both sides from said electrode shaft, wherein the contact surface is arranged on the side of the holding bracket lying opposite to the electrode shaft. As a result of this design of the electrode, the latter can be placed between the internal jugular vein and the common carotid artery in such a way that the electrode shaft lies in the interspace between these two blood vessels and the holding bracket rests against the vein with its one side and against the artery with its other side and presses against the vagus nerve to be stimulated with its front side, on which the contact surface is situated and which faces away from the electrode shaft.

Although this form of electrode is suitable for intraoperative nerve stimulation during an operation, it still has a few weaknesses in respect of reliable fixing of the electrode and secure contacting of the vagus nerve. Hence, as a result of the predetermined shape of the electrode, anatomical variances can be matched only within the scope of the predetermined contour of the electrode body with the T-shaped design. Moreover, the relatively small contact surface in conjunction with contact force, which is only exerted to a medium extent by means of the blood vessels via the holding bracket, cannot ensure reliable electrical contacting in every intraoperative situation.

The document DE 10 2007 036 862 A1, which likewise originates from the applicant, proposed an electrode for intraoperative stimulation of the vagus nerve, which electrode is characterized by a contact strip, which can be looped around the nerve to be stimulated and closed to form a closed loop, is made of an elastic, biocompatible material, has at least one electrically conductive contact surface which faces the nerve and can, by means of a signal-transmission means, be connected to a signal transmitter and/or a signal recorder. The contact strip can be applied to the nerve to be stimulated in a fashion similar to a cable tie and can remain on said nerve as a cuff during the operation and be continuously actuated with stimulation signals.

Although this achieves reliable electrical contacting with the vagus nerve, applying and detaching the electrode requires much practice and skill of the operator since the position of the vagus nerve is covered behind the blood vessels in the operation field and said vessels must not be injured when the nerve is exposed, as required for applying the electrode. Nor may the contact strip be pulled together too tightly so as to avoid a crush trauma of the nerve.

### SUMMARY OF THE INVENTION

Hence, an object on which the present invention is an improved electrode for intraoperative monitoring of the vagus nerve to the effect that, firstly, uncomplicated applying and detaching of the electrode prior to and after the operation and, secondly, a stable seat of the electrode and reliable contacting of the nerve during the intervention are ensured.

In conjunction with the features of the preamble, this object and others are achieved by virtue of the fact that the electrode holder, at its top end has a first portion with a plastically deformable material such that, during the application, the electrode holder can, with the first portion, be formed onto a vessel wall of a first blood vessel extending parallel to the vagus nerve and encompasses this blood vessel in order to affix the electrode in a hook-shaped fashion and wherein the electrode holder has a second portion, which, with an opposing curvature to the first portion, is placed around the vagus nerve in a loop-like fashion and can be passed through between the first blood vessel and a second blood vessel which extends parallel thereto.

As result of working a plastically deformable material into a first portion situated at the top end of the electrode holder, the electrode holder can, in a forming fashion, be matched to the anatomical structure of the organs. In an advantageous fashion, the electrode holder is, in its top region, bent around the neighboring blood vessel—the common carotid artery—in such a way that the latter is encompassed along its circumference by the end section of the electrode holder in a hook-like fashion. As a result, the electrode experiences a secure hold, even during the operation.

In a second portion, situated in the central to bottom-end region of the electrode holder, the electrode holder is, according to the invention, looped around the vagus nerve with an opposite curvature to the first portion, wherein the then interior contact surface(s) touches/touch the nerve. As a result of the tensile force exerted by the hook-like mounting of the electrode holder, the at least one contact surface presses against the vagus nerve and ensures reliable stimulation.

In the further course toward the bottom end, the electrode holder is guided through between the common carotid artery and a second blood vessel—the internal jugular vein—and thereby additionally supported on the side lying opposite to the hook-shaped enclosure.

Thus, the electrode is applied without complication in such a way that the electrode holder is guided under the vagus nerve from the side of the internal jugular vein and then placed around the vessel wall of the common carotid artery by means of the top of the electrode.

Thus, in the solution according to the invention, a large circumferential angle around the common carotid artery ensures high mechanical stability and, as a result therefrom, enables reliable electrical contacting in conjunction with a likewise large circumferential angle about the vagus nerve—in the case of an appropriate design of the contact surface.

In a preferred embodiment of the electrode, the plastically deformable material includes a strip-shaped metal film worked into the electrode holder. As a result of the strip-shaped metal film, the plastic deformability of the electrode body for forming the hook-shaped holder of the electrode is achieved in a simple fashion. After the electrode has been introduced, the metal strip is bent around the vessel wall of the common carotid artery and thereby imparts a secure hold, which can, after the operation has taken place, be released again without problems.

In order to exploit the contact region which can be used by looping around the vagus nerve, the contact surface(s) preferably extends/extend parallel to the longitudinal extent of the electrode holder in a strip-shaped fashion. Such an embodiment of the contact surface(s) allows a reliable continuous stimulation, even in the case of small movements of the electrode.

The stimulation is preferably brought about by means of a connection cable as signal-transmission medium, which is routed away from the electrode holder at a bottom end of the electrode holder or substantially centrally between the bend around the blood vessel and the contact surface to the vagus nerve. A wired transmission of the signals is less susceptible to radiofrequency interference and thereby contributes to reliable signal transmission of the stimulation pulses or the response signals thereof. Depending on the conditions in the operation, the connection cable can in this case be routed away from the electrode holder, either at the bottom end or approximately centrally.

It is found to be advantageous that the elastic holder material, from which the electrode holder is made, is electrically non-conductive medical silicone. In conjunction with a metal film, which is worked in as a plastically deformable material, the holder material assumes the function of an insulation layer for the metal film with respect to the contact surfaces. The metal film itself is preferably produced from medical stainless steel.

An advantageous development of the electrode can have a larger first portion with worked-in plastically deformable material, which extends into the second portion. A region of plastically deformable material extended thus enables even stronger attachment of the electrode, particularly in the region of the contact surfaces. This in turn increases the reliability of the signal transmission from the electrode contact surfaces to the nerve.

Moreover, the object on which the invention is based, in conjunction with the features of the preamble, is also achieved by virtue of the fact that the electrode holder, at least at the top end thereof, has a pre-formed section with a bend such that, during the application, the electrode holder encompasses a vessel wall of a first blood vessel extending parallel to the vagus nerve in order to affix the electrode in a hook-shaped fashion and wherein the electrode holder can, in the further course with an opposing curvature, be placed around the vagus nerve in a loop-like fashion and passed through between the first blood vessel and a second blood vessel which extends parallel thereto.

In contrast to the technical teaching claimed above, the electrode holder of this solution has, at least at the top end thereof, a pre-formed section with a bend, the radius of curvature of which is already matched to the circumference of the common carotid artery extending adjacent thereto. This curved pre-formed section is placed around the vessel wall of the artery in a hook-like fashion during application—it is, as it were, hooked in—and thus fixes the inserted electrode. In the further course, the electrode holder is, like in the above-described electrode, guided around the vagus nerve in a loop-like fashion with an opposite curvature such that reliable contacting is ensured in the case of an embodiment of the contact surfaces as strip-shaped conductor tracks extending in parallel to the longitudinal extent of the electrode holder. Toward the bottom end, the electrode holder is placed in a known fashion between the common carotid artery and the internal jugular vein and additionally supported thereby.

The stimulation is brought about in the same fashion as in the case of the electrode made of completely elastic material, by means of a connection cable with an output from the electrode holder arranged at one end or centrally.

The pre-formed section can consist of a substantially rigid material and, in a preferred embodiment, be embodied as an injection-molded part. Such an embodiment is particularly suitable for industrial-scale manufacturing.

These and other objects, aspects, examples, features and advantages, embodiments of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which form a part hereof and wherein:

FIG. 1 shows a top view of a first embodiment of an electrode according to the invention with a long metal film,

FIG. 2 shows the subject of FIG. 1 in the longitudinal section along the cut surface II-II,

FIG. 3 shows a top view of a second embodiment of an electrode according to the invention with a short metal film,

FIG. 4 shows a top view of a third embodiment of the electrode according to the invention with a centrally arranged signal output,

FIG. 5 shows the second embodiment of the electrode according to the invention during application in the vessel/nerve sheath,

FIG. 6 shows the third embodiment of the electrode according to the invention during application in the vessel/nerve sheath,

FIG. 7 shows an embodiment of an electrode according to the invention with a pre-formed section during application in the vessel/nerve sheath.

### DESCRIPTION OF PREFERRED EMBODIMENTS

Referring now to the drawings wherein the showings are for the purpose of illustrating preferred and alternative embodiments of the invention only and not for the purpose of limiting the same, FIG. 1 reproduces a first embodiment of an electrode 2 according to certain aspects of the invention. In this top view, an electrode holder 4 can be seen as a main element of the electrode 2. The electrode holder 4 is formed by an elastic material, which is preferably electrically non-conductive medical silicone.

A plastically deformable material in the form of a thin the metal film 8 is embedded into this holder material 4 in a first portion 6 which faces the top end 5 of the electrode holder 4. The metal film 8 can easily be formed and can therefore, in a simple fashion, be matched by bending to the anatomical structures of the body organs—in this case to the vessel wall of the common carotid artery (see also FIG. 5 and FIG. 6). As a result of using electrically non-conductive medical silicone as holder material 4, there is no need to introduce a special electrical insulation layer to separate the metal film 8 from other electrical components of the electrode carrying potential. Medical stainless steel is found to be particularly suitable for the metal film 8.

Three strip-shaped electrical contact surfaces 12, which extend parallel to the longitudinal extent of the electrode holder 4, are arranged on an outer side of the electrode holder 4 in a second portion 10. The first portion 6, into which the metal film 8 has been worked, extends into the second portion 10 holding the contact surfaces 12 such that the two portions 6 and 10 cover one another in this first embodiment.

In the direction of the bottom end 11 of the electrode holder 4, the contact surfaces 12 are connected to cable cores 14, which form the connection cable 16 that serves as signal-transmission medium 16. At the bottom end 11, the connection cable 16 is routed away along the electrode holder 4 and connected to a signal transmitter and/or a signal receiver (not illustrated).

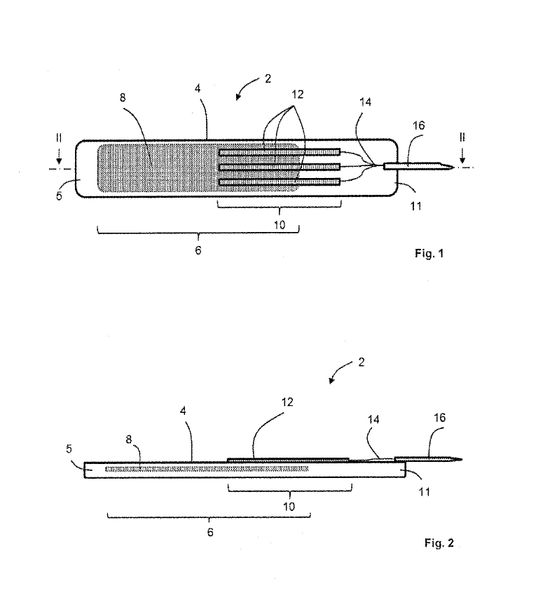

FIG. 2 shows the first embodiment, illustrated in FIG. 1, in the longitudinal section along the cut surface II-II. It can clearly be seen that, as a result of the first portion 6 being covered by the second portion 10, the metal film 8 situated in the first portion 6 is arranged below the contact surfaces 12 situated in the second portion 10. Here, the electrode holder 4 made of electrically non-conductive medical silicone surrounds the embedded metal film 8 and forms an electrically insulating separation layer, in particular with respect to the contact surfaces 12.

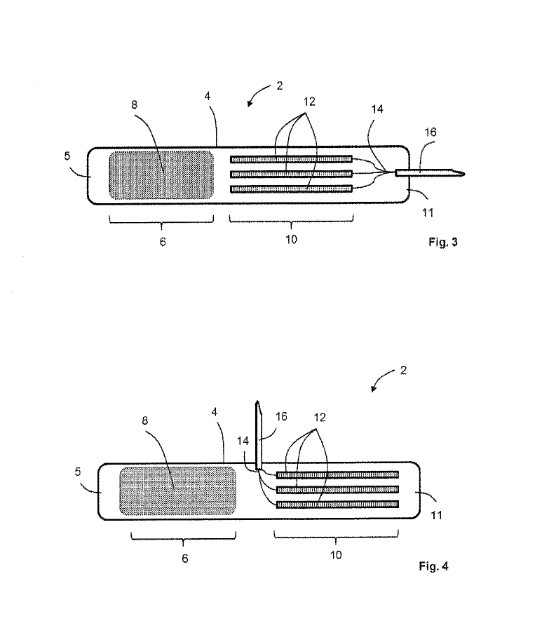

FIG. 3 shows a second embodiment of the electrode 2 according to the invention, in which the first portion 6, into which the metal film 8 has been worked, does not extend into the second portion 10 which carries the contact surfaces 12. Hence, the difference to the first embodiment merely consists in a shortened length of the metal film 8, which therefore restricts the plastically deformable portion 6 to a region substantially comprising the top-side half of the electrode holder 4.

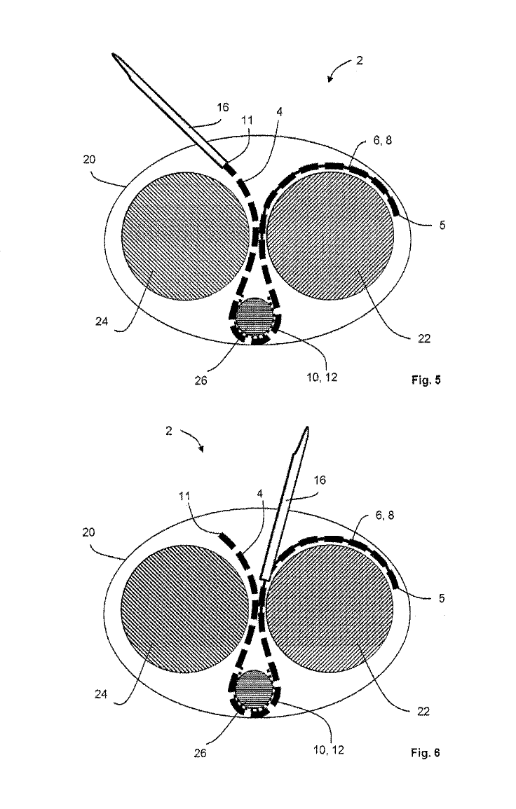

A further, third embodiment of the electrode 2 according to the invention is depicted in FIG. 4. In this embodiment, the metal film 8, just like the electrode 2 disclosed in FIG. 3, has a shortened length; however, the cable cores 14 and hence the connection cable 16 are not routed away along the bottom end 11 of the electrode holder 4 in this third embodiment. Rather, the connection between contact surfaces 12 and the connection cable 16 is aligned toward the center of the electrode holder 4. This embodiment can take into the account special spatial conditions during the operation. Except for the alignment of the contact surfaces 12, this embodiment otherwise corresponds to the embodiment of an electrode 2 with a short worked-in metal film 8, as described in FIG. 3.

FIGS. 5 to 7 relate to the application of the electrode 2 according to the invention during a thyroid operation. Illustrated schematically is a respective embodiment of the electrode 2 in conjunction with the anatomy of the vessel/nerve sheath 20 (carotid sheath). Illustrated in each case is, looking toward the skull on the right-hand body side, the position of the common carotid artery 22, the internal jugular vein 24 and the vagus nerve 26 to be stimulated.

With reference to FIG. 3, FIG. 5 shows the position of the electrode with shortened length of the metal film 8 during a surgical intervention. During the application, the operator guides the electrode 2 under the vagus nerve 26 from the side of the internal jugular vein 24 in order then to support it in a hook-shaped fashion on the common carotid artery 22. This results in a curved loop-like enclosure, with the curvature opposite to that of the first portion 6, of the vagus nerve 26 by the second portion 10 of the electrode holder 4 holding the contact surfaces 12. In conjunction with the strip-shaped contact surfaces 12 arranged parallel to the longitudinal extent of the electrode holder 4, the large enclosure angle ensures reliable contacting of the nerve 26 to be stimulated. The support on the common carotid artery 22 is brought about by forming a bend from the metal film 8 embedded into the electrode holder 4 in the region of the first portion 6. As a result, the electrode holder 4 can be matched plastically at its top end 5 to the vessel wall of the common carotid artery 22. The connection cable 16 is, in accordance with FIG. 3, routed away at the bottom end 11 of the electrode holder 4.

FIG. 6 illustrates the third embodiment in accordance with FIG. 4, with short metal film 8 and the connection cable 16 being routed away centrally. Depending on the operation surroundings—e.g. in the case of cramped spatial conditions in the region of the internal jugular vein 24—such a central position of the cable output between stimulation surface and bend around the common carotid artery 22 may be advantageous.

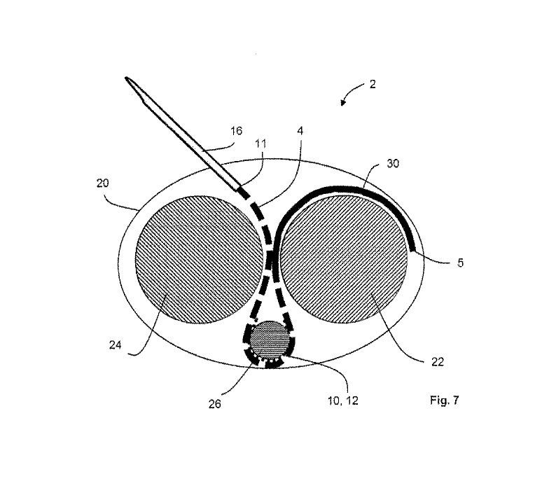

FIG. 7 illustrates the position of a further electrode 2 according to the invention, in which the electrode holder 4 has, at the top end 5 thereof, a pre-formed section 30 with a bend, the radius of curvature of which being matched to the circumference of the common carotid artery 22. Like an electrode which consists entirely of elastic material, the electrode 2 can be inserted into the vessel/nerve sheath 20 and guided around the vagus nerve 26 in a loop-like manner. This electrode 2 is characterized by virtue of the fact that the curved section 30, by means of which the electrode 2 is, at the top end 5 thereof, “hooked” into the common carotid artery 22, is already pre-formed from a flexibly elastic material. Here, the pre-formed region can extend beyond the bend around the common carotid artery 22 into the second portion 10 holding the contact surfaces 12.

Using the invention, an electrode for intraoperative stimulation of the vagus nerve is created, having an electrode holder, which has at least one contact surface and an approximately strip-shaped design, which electrode holder can be applied to the vagus nerve in such a way that the contact surface touches the nerve on at least part of the circumference thereof, wherein, according to the invention, the embodiment is made such that the electrode holder has a first curved section which is matched or can be matched to the vagus nerve and on the inner side of which the at least one contact surface is arranged, and that the electrode holder has at least a second section, which is formed or can be formed approximately in a hook-like manner opposite to the curvature of the first section and is matched or can be matched to a blood vessel which extends neighboring the vagus nerve.

The invention is not restricted to the illustrated exemplary embodiments; rather, various modifications or extensions emerge without departing from the scope of the invention. Thus, the whole electrode 2 can be made of a pre-formed part, wherein it preferably consists of a flexibly elastic material which, in accordance with FIGS. 5 to 7, is matched in terms of its form to the anatomy of the vessel/nerve sheath 20 and has the illustrated advantageous profile of curvature, but which can be bent when the electrode is applied to the vagus nerve and therefore can easily be applied and also removed again.

Further, while considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-9. (canceled)

10. An electrode for intraoperative stimulation of the vagus nerve, the electrode comprising an electrode holder with a strip-shaped design and at least one electrically conductive contact surface, the at least one electrically conductive contact surface being connectable via a signal-transmission medium to at least one of a signal transmitter and a signal receiver and configured to touch an associated vagus nerve on part of its circumference during the application, the electrode holder further including a first, plastically deformable portion, which, during application of the electrode holder can be formed onto an associated vessel wall of an associated first blood vessel extending parallel to the associated vagus nerve and encompasses the first associated blood vessel in order to affix the electrode in a hook-shaped fashion, the electrode holder further including a second portion, which, with an opposing curvature to the first portion, can be placed around the associated vagus nerve in a loop-like fashion and can be passed through between the associated first blood vessel and a second associated blood vessel which extends parallel to the first associated blood vessel.

11. The electrode as claimed in claim 10, wherein the first plastically deformable portion has a strip-shaped metal film within the electrode holder.

12. The electrode as claimed in claim 11, wherein the electrode holder is formed by an electrically non-conductive medical silicone.

13. The electrode as claimed in claim 10, wherein the electrode holder is formed by an electrically non-conductive medical silicone.

14. The electrode as claimed in claim 11, wherein the metal film is formed by a medical stainless steel.

15. The electrode as claimed in claim 12, wherein the metal film is formed by a medical stainless steel.

16. The electrode as claimed in claim 10, wherein the first plastically deformable portion extends at least as far as the at least one electrically conductive contact surface.

17. The electrode as claimed in claim 10, wherein the at least one electrically conductive contact surface extends parallel to the longitudinal extent of the electrode holder in a strip-shaped fashion.

18. The electrode as claimed in claim 10, wherein the signal-transmission medium is a connection cable, which is routed away from the electrode holder at one end of the electrode holder.

19. The electrode as claimed in claim 10, wherein the signal-transmission medium is a connection cable, which is routed away from the electrode holder in a transition region between the bend around the associated first blood vessel and the contact surface to the associated vagus nerve.

20. The electrode as claimed in claim 10, wherein the electrode holder is an injection-molded part.

21. The electrode as claimed in claim 10, wherein the electrode holder is formed by a flexibly elastic material.

22. An electrode for intraoperative stimulation of the vagus nerve, the electrode comprising an electrode holder with a strip-shaped design and made of an elastic material, the electrode further comprising at least one electrically conductive contact surface, the at least one electrically conductive contact surface being connectable via a signal-transmission medium to at least one of a signal transmitter and a signal receiver and touching an associated vagus nerve on part of its circumference during the application, the electrode holder having a first section having an approximately hook-shaped form with a bend, which, during the application of the electrode, extends over an associated vessel wall of an associated first blood vessel extending parallel to the associated vagus nerve in order to affix the electrode, the electrode holder further including a second section with an opposing curvature, by way of which the second section can be placed around the associated vagus nerve in a loop-like fashion and passed through between the associated first blood vessel and an associated second blood vessel which extends parallel to the associated first blood vessel.

23. The electrode as claimed in claim 22, wherein the at least one electrically conductive contact surface extends parallel to the longitudinal extent of the electrode holder in a strip-shaped fashion.

24. The electrode as claimed in claim 22, wherein the signal-transmission medium is a connection cable, which is routed away from the electrode holder at one end of the electrode holder.

25. The electrode as claimed in claim 22, wherein the signal-transmission medium is a connection cable, which is routed away from the electrode holder in the transition region between the bend around the associated first blood vessel and the contact surface to the associated vagus nerve.

26. The electrode as claimed in claim 22, wherein the electrode holder is an injection-molded part.

27. The electrode as claimed in claim 22, wherein the electrode holder is formed by a flexibly elastic material.
